(19) United States
(12) Patent Application Publication
(10) Pub. No.: US 2025/0352568 A1
(43) Pub. Date: Nov. 20, 2025

(54) COMPOSITION OF MANGIFERIN AND PROCESS OF PREPARATION THEREOF

(71) Applicant: ZEUSHYGIA LIFESCIENCES PVT LTD., Hyderabad (IN)

(72) Inventors: Shankaranarayanan JEYAKODI, Hyderabad (IN); Arunkanth Krishnakumaran NAIR, Hyderabad (IN)

(21) Appl. No.: 19/208,983

(22) Filed: May 15, 2025

(30) Foreign Application Priority Data
May 15, 2024 (IN) 202241065502

(51) Int. Cl. A61K 31/7048; A61K 9/48; A61K 36/22; A61P 25/28

(52) U.S. Cl. CPC A61K 31/7048; A61K 9/4866; A61K 36/22; A61P 25/28

(57) ABSTRACT

The present invention relates to a proprietary encapsulation technology where in a composition comprising mangiferin will maintain in-situ alkaline environment and minimizes the precipitation issues of mangiferin in the acidic environment. A composition of mangiferin has been disclosed. A natural hydrophilic plant-based carrier along with a selected alkalizer is being embedded in the encapsulation matrix which subsequently acts as a platform to ensure the alkaline environment with better solubility of bioactive across the physiological pH range.

### FIELD OF THE INVENTION

The present disclosure generally relates to a composition comprising mango bark extract rich in mangiferin acting as a catechol-o-methyltransferase (COMT) inhibitor, having enhanced physicochemical properties ensuring better therapeutic properties. This enhancement has a profound impact on improving working memory, selective attention and executive function. Further the present invention relates to a process for preparing the composition.

### BACKGROUND OF THE INVENTION

Mangifera indica, also known as the mango, has been a valued herb in Ayurvedic and indigenous medical systems for over 4,000 years. One of the most popular tropical fruits, the mango, possesses strong antioxidant, anti-lipid peroxidation, immunomodulating, cardiotonic, hypotensive, wound healing, antidegenerative, and antidiabetic activities due to its presence of polyphenolic antioxidants and glucosyl xanthones [Shah et al., 2010].

Chronic accumulation of lipopolysaccharide (LPS) can induce neuroinflammation, ultimately leading to cognitive deficits. Heme oxygenase 1, a key enzyme in antioxidant and anti-inflammatory pathways, is downregulated during this process. Mangiferin supplementation was found to decrease LPS-induced IL-6 production and upregulate heme oxygenase-1 expression in the hippocampus, suggesting its potential as a neuroprotective agent [Fu et al., 2015].

Mangiferin, an antioxidant and anti-inflammatory agent, was shown to prevent Sleep Deprivation (SD)-induced behavioral and neurochemical changes in mice. Sleep deprivation in mice significantly impaired learning and cognition, as evidenced by tests like the Morris water maze and novel object recognition task. Pretreatment with Mangiferin provided protection against these SD-induced neurobehavioral and neurochemical changes.

Additionally, Mangiferin offered beneficial effects against the increase in pro-inflammatory cytokine levels in the periphery and brain, reduced oxidative stress, and restored the decreased levels of BDNF (Brain-Derived Neurotrophic Factor) in both plasma and hippocampus [Feng et al., 2017].

Recent studies investigating brain-relevant enzyme inhibition have shown that mangiferin significantly inhibited catechol-O-methyltransferase (COMT), the enzyme responsible for degrading catecholamine neurotransmitters. COMT's catabolic pathway is particularly prevalent in brain tissue with low levels of catecholamine reuptake transporters. COMT inhibition primarily affects dopaminergic function in the prefrontal cortex and hippocampus, potentially leading to improvements in working memory, selective attention, and executive function [López-Ríos et al., 2020]. Long-Term Potentiation (LTP) can be conceptualized as the persistent strengthening of synaptic connections and is a major cellular mechanism behind learning and memory.

Further, Mangiferin and Mangifera indica extract were reported to increase LTP [López-Ríos et al., 2020]. Mangiferin ameliorated the scopolamine-induced learning deficits in mice, which was thought to be partly due to its inhibitory mechanism against acetylcholinesterase [Jung et al., 2009]. Mangiferin was reported to exhibit its neuroprotective effects by multiple mechanisms including its antioxidant, acetylcholinesterase inhibition, and lipoxygenase inhibition [Sethiya and Mishra, 2014].

However, Mangiferin is considered to have pH dependent solubility and the solubility increases towards alkaline pH range. Studies reveal that Mango bark extract (mangiferin) has good solubility in phosphate buffer (pH 6.8) and is slightly water soluble also (0.16 mg/mL ca., 37° C.). According to the biopharmaceutic classification system (BCS), mangiferin belongs to class IV, therefore, peroral delivery is partially hindered due to its poor intestinal permeability.

The clinical efficacy of mangiferin is severely limited due to its low bioavailability. The major reasons behind low bioavailability are low solubility in gastric fluids (i.e., Acidic pH), shorter half-life and rapid clearance from GIT.

Reference is made to “Increased absorption of mangiferin in the gastrointestinal tract and its mechanism of action by absorption enhancers in rats” authored by XiaoliWang, Yuechen Gu, Tianyang Ren,Bin Tian,Yu Zhang,Lingkuo Meng,Xing Tang. This document describes that in order to improve the oral absorption of mangiferin, potential enhancers, including TPGS, sodium deoxycholate and Carbopol 974P, could be used. The study shows that the increased oral absorption by the three co-excipients was in the order of Carbopol 974P>sodium deoxycholate>TPGS.

Further reference is made to “Mangiferin: a review of dietary sources, absorption, metabolism, bioavailability, and safety” authored by Suhuan Mei,ManivelPerumal,Maurizio Battino34,David D Kitts,Jianbo Xiao,Haile Ma,Xiumin Chen, DOI:10.1080/10408398.2021.1983767. This study envisages that recent research has led to the development of novel technologies to encapsulate mangiferin in nano/microparticle carrier systems as well as generate mangiferin derivatives to improve solubility and bioavailability.

Another reference is made to “Nanotechnology-Based Drug Delivery Approaches of Mangiferin: Promises, Reality and Challenges in Cancer Chemotherapy”, authored by Muhammad Sarfraz,Abida Khan,Gaber El-Saber Batiha,Muhammad FurqanAkhtar,Ammara Saleem,Basiru Olaitan Ajiboye,Mehnaz Kamal,Abuzer Ali,Nawaf M.Alotaibi, Shams Aaghaz,Muhammad Irfan Siddique, andMohd Imran; doi:10.3390/cancers15164194. This study shows that Nano-drug delivery systems have attempted to deliver the drugs at the desired site at a desired rate in desired amounts. Many researchers have explored various nanotechnology-based approaches to provide effective and safe delivery of mangiferin for cancer therapy. Nanoparticles were used as carriers to encapsulate mangiferin, protecting it from degradation and facilitating its delivery to cancer cells. They have attempted to enhance the bioavailability, safety and efficacy of this very bioactive using drug delivery approaches.

However, although several attempts have been made to improve the solubility of mangeferin in gastric (acidic) region, improving the solubility in lower acidic pH and subsequently improving the clinical efficacy could not be achieved.

The composition in accordance to the present invention comprises mango extract rich in mangiferin acting as a catechol-o-methyltransferase (COMT) inhibitor, processed using a proprietary encapsulation technology improves the lower acidic pH solubility and eventually the clinical efficacy. This enhancement has a profound impact on improving working memory, selective attention and executive function.

Mangiferin's potential acetylcholinesterase (AChE) inhibition fosters heightened acetylcholine activity. This holistic effect bolsters focused attention and memory consolidation, contributing to its cognitive benefits.

The proposed invention is further aimed to address the low acidic solubility & subsequent clinical efficacy issues of mangiferin.

### OBJECT OF THE INVENTION

The aim of the present invention is to provide a composition comprising mango extract rich in mangiferin with improved solubility and clinical efficacy improving working memory, selective attention and executive function.

The object of present invention is to provide a composition comprising mango extract rich in mangiferin acting asacetylcholinesterase (AChE) inhibition fostering heightened acetylcholine activity thereby enhancing focused attention and memory consolidation, contributing to its cognitive benefits.

Another object of the present invention is to provide a composition comprising mango extract rich in mangiferin with improved acidic solubility & subsequent enhanced clinical efficacy.

Another object of present invention is to address the low acidic solubility & subsequent clinical efficacy issues of mangiferin.

Yet another object of present invention is to enrich the in-vitro-in-vivo characteristics of mangiferin.

### SUMMARY OF THE INVENTION

In an embodiment of present invention, there is provided a composition comprising mango extract rich in mangiferin encapsulated within a hydrophilic carrier matrix comprising an alkalizer embedded therein, wherein the composition comprising from about 50% to about 70% mangiferin encapsulated within from about 20% to about 40% a hydrophilic carrier matrix comprising about 1% to about 5% an alkalizer embedded therein.

In another embodiment, said hydrophilic carrier is a natural hydrophilic plant-based carrier.

In another embodiment, said hydrophilic carrier is selected from gum arabic powder, xanthan gum, modified starch powder, and combinations thereof.

In yet another embodiment, said hydrophilic carrier is gum Arabic powder.

In a further embodiment, said alkalizer is selected from calcium oxide, calcium chloride, sodium carbonate, lithium carbonate, potassium carbonate, sodium hydrogen carbonate, lithium hydrogen carbonate, potassium hydrogen carbonate, disodium hydrogenphosphate, sodium citrate, dipotassium hydrogenphosphate, sodium acetate, sodium lauryl sulphate, polacriline potassium, calcium hydroxide, meglumine, magnesium aluminometasilicate and magnesium oxide or combination thereof.

In a preferred embodiment, the alkalizer is magnesium oxide with at least one other alkalizer as exemplified above.

In another embodiment, said composition further comprises an emulsifier.

According to an embodiment, said composition comprises about 0.5% to about 5% (w/w) of an emulsifier.

According to another embodiment, the emulsifier is selected from Polysorbate 80, sunflower lecithin and combinations thereof.

According to another embodiment, the composition further comprises an additive.

According to yet another embodiment, the additive, if present is present in the form selected from thickeners, fragrances, pearlescent agents, preservatives, anionic or non-ionic or cationic or amphoteric polymers, proteins, protein hydrolysates, fatty acids, vitamins, panthenol, vegetable, mineral or synthetic oils, gelling agents, antioxidants, solvents, fragrances, fillers, colouring materials and combinations thereof.

According to yet another embodiment, the additive if present is less than 50% by weight with respect to the total weight of the composition.

According to yet another embodiment, the composition is a bio-accessible composition.

According to another aspect of present invention, there is disclosed a process for preparing a composition of mango extract rich in mangiferin comprising the steps of:
a. dissolving mango extract rich in mangiferin in purified water;
b. dissolving separately a hydrophilic carrier and an alkalizer in purified water;
c. mixing solutions of step a. and b. and passing it through a high pressure homogenizer to form uniform dispersion.
d. spray drying the dispersion of step c.

According to an embodiment of present invention, passing said mixed solution through homogenizer in step c is performed preferably for about 15-30 minutes.

According to another embodiment of present invention, passing said mixed solution through homogenizer in step c is performed more preferably for about 15-25 minutes.

According to another embodiment of present invention, passing said mixed solution through homogenizer in step c is performed most preferably for about 15-20 minutes.

According to another embodiment of present invention, said spray drying in step d. is performed at an inlet temperature from about 100° C. to about 180° C. and an outlet temperature from about 30° C. to about 90° C., preferably inlet temperatures from about 110 to about 150° C. and outlet temperatures from about 30 to about 60° C.

According to an embodiment of present invention, said spray drying in step d. is performed preferably at an inlet temperature from about 110° C. to about 150° C. and an outlet temperature from about 30° C. to about 60° C.

According to yet another embodiment of present invention, said hydrophilic carrier is a natural hydrophilic plant-based carrier.

According to further embodiment of present invention, said hydrophilic carrier is selected from gum arabic powder, xanthan gum, modified starch powder, and combinations thereof.

In an embodiment, said hydrophilic carrier is gum Arabic powder.

In another embodiment, said alkalizer is selected from calcium oxide, calcium chloride, sodium carbonate, lithium carbonate, potassium carbonate, sodium hydrogen carbonate, lithium hydrogen carbonate, potassium hydrogen carbonate, disodium hydrogenphosphate, sodium citrate, dipotassium hydrogenphosphate, sodium acetate, sodium lauryl sulphate, polacriline potassium, calcium hydroxide, meglumine, magnesium aluminometasilicate and magnesium oxide or combination thereof.

In a preferred embodiment, the alkalizer is magnesium oxide with at least one other alkalizer as exemplified above.

In yet another embodiment, the mangiferin in step a. is dissolved in 80% of purified water.

In a further embodiment, the carrier in step b. is dissolved in 20% of purified water followed by dissolving in the said solution an optional further carrier, the alkaliser and the emulsifier.

In another embodiment, the amount of mangiferin per dosage form is about 300 mg.

In an embodiment, wherein the composition is in oral dosage form.

In further embodiment, the oral dosage form is selected from a buccal strip, bar, gummies, a chewing gum, a tablet, a capsule, an emulsion, a suspension, an oral spray, effervescent, dissolvable granules or powder, a sachet, and a clear beverage.

In another embodiment, the buccal strip is an orally dissolvable or dispersible buccal strip.

In another embodiment, the oral dosage form is a tablet.

In yet another embodiment, the oral dosage form is a capsule.

In yet another embodiment, the mangiferin acts as a catechol-o-methyltransferase (COMT) inhibitor.

According to another aspect of present invention, there is provided a therapeutic method for improving working memory, selective attention and executive function and enhancement of cognitive abilities comprising the step of administering the composition as discussed above.

According to an embodiment of present invention, the dosage of the mangiferin administered is about 300 mg per day.

According to yet another embodiment, the dosage of the mangiferin administered is for about 7 days.

### Advantageous Effects

The features and advantages of the present disclosure may be summarized as follows:
(a) The present disclosure provides a proprietary composition to boost the Physico chemical properties of mangiferin. This enhancement has a profound impact on improving working memory, selective attention and executive function.
(b) The mangiferin of the illustrated composition functioning synergistically demonstrates potential acetylcholinesterase (AChE) inhibition fosters heightened acetylcholine activity. This holistic effect bolsters focused attention and memory consolidation, contributing to its cognitive benefits.
(c) The composition of the present disclosure is a composition of mango extract rich in mangiferin having improved acidic solubility & subsequent enhanced clinical efficacy.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### BRIEF DESCRIPTION OF THE ACCOMPANYING DRAWINGS

FIG. 1 illustrates % changes/Improvement in Cognitive abilities—Post Supplementation of Present Invention (Stadice) compared to Placebo. Mental speed assessed by DSST, focused attention assessed by colour trails test 1 (CTT1), sustained attention assessed by errors reduction in Digit Vigilance test, response inhibition assessed by Stroop Test, AVLT immediate recall and AVLT delayed recall by Auditory Verbal Learning Tests (AVLT).

### DETAILED DESCRIPTION OF THE INVENTION

The articles “a” and “an,” as used herein, mean one or more when applied to any feature in embodiments of the present invention described in the specification and/or claims. The use of “a” and “an” does not limit the meaning to a single feature unless such a limit is specifically stated. The article “the” preceding singular or plural nouns or noun phrases denotes a particular specified feature or specified features and may have a singular or plural connotation depending upon the context in which it is used. The adjective “any” means one, some, or all indiscriminately of whatever quantity.

The term “bio-accessible composition” refers to the composition having enhanced bioaccessibility. Bio-accessibility is defined as the percentage of the bioactive compounds (BCS) solubilized after intestinal dialysis step; Bio-accessibility index defines the proportion of the bioactive compound that could become available for absorption into the blood system. In present case, the composition of mangiferin improves the solubility of otherwise insoluble mangiferin in gastric (acidic) region, improving the bio-accessibility of the same.

As used herein, the phrase “Mangiferin” refers to a xanthone that have anticancer, antioxidant, antimutagenic, antidiabetic, antibacterial, and anti-inflammatory properties, found in high plants and in any material from the Mangifera indica (mango) plant, including, but not limited to, stem bark, fruits, leaves and combinations thereof.

The inventors of the present invention have unexpectedly found that ‘Mangiferin’ acting as a catechol-o-methyltransferase (COMT) inhibitor acts synergistically and effectively when the solubility was enhanced using the processing technology of present invention. This enhancement has a profound impact on improving working memory, selective attention and executive function.

Additionally, mangiferin's potential acetylcholinesterase (AChE) inhibition fosters heightened acetylcholine activity. This holistic effect bolsters focused attention and memory consolidation, contributing to its cognitive benefits.

Various embodiments of the present invention disclosed herein relate to herbal compositions for enhancing alertness, attention, concentration, and/or memory, comprising mangiferin. Mangiferin is found in several botanicals, including extracts of mango fruit, mango peel, mango bark, mango leaf, as well as in Cyclopia species (Honeybush tea) and species in the genus Salacia. Mangiferin has acetylcholinesterase inhibiting activity, an activity useful in improving cognitive function in Alzheimer's disease.

Mangiferin, a xanthonoid, is a natural phenolic compound formed from the xanthone backbone. Mangiferin is preferably extracted from a plant containing mangiferin.

The present invention relates to a composition comprising mango extract rich in mangiferin encapsulated within a hydrophilic carrier matrix comprising an alkalizer embedded therein.

As used herein, the term “encapsulation” refers to a carrier matrix formed by emulsifying mangiferin within a hydrophilic carrier wherein said mangiferin is entrapped within said carrier matrix.

In The phrase “carriers” generally refers to biocompatible tools for the transport of molecules for pharmaceutical, cosmetic, and nutraceutical applications. As used herein the mangiferin is encapsulated within a natural hydrophilic carrier matrix.

The present invention resides in a composition comprising mango extract rich in mangiferin encapsulated within a hydrophilic carrier matrix comprising an alkalizer embedded therein.

In an embodiment, the composition comprises from about 50% to about 70% mangiferin encapsulated within from about 20% to about 40% a hydrophilic carrier matrix comprising about 1% to about 5% an alkalizer embedded therein.

In another embodiment, said hydrophilic carrier is a natural hydrophilic plant-based carrier.

In another embodiment, said hydrophilic carrier is selected from gum arabic powder, xanthan gum, modified starch powder, and combinations thereof.

In yet another embodiment, said hydrophilic carrier is gum Arabic powder.

In a further embodiment, said alkalizer is selected from calcium oxide, calcium chloride, sodium carbonate, lithium carbonate, potassium carbonate, sodium hydrogen carbonate, lithium hydrogen carbonate, potassium hydrogen carbonate, disodium hydrogenphosphate, sodium citrate, dipotassium hydrogenphosphate, sodium acetate, sodium lauryl sulphate, polacriline potassium, calcium hydroxide, meglumine, magnesium aluminometasilicate and magnesium oxide or combination thereof.

In a preferred embodiment, the alkalizer is magnesium oxide with at least one other alkalizer as exemplified above.

In another embodiment, said composition further comprises an emulsifier.

According to an embodiment, said composition if present, is present in an amount about 0.5% to about 5% (w/w) of an emulsifier.

According to another embodiment, the emulsifier is selected from Polysorbate 80, sunflower lecithin and combinations thereof.

According to another embodiment, the composition further comprises an additive.

According to yet another embodiment, the additive, if present is present in the form selected from thickeners, fragrances, pearlescent agents, preservatives, anionic or nonionic or cationic or amphoteric polymers, proteins, protein hydrolysates, fatty acids, vitamins, panthenol, vegetable, mineral or synthetic oils, gelling agents, antioxidants, solvents, fragrances, fillers, coloring materials and combinations thereof.

According to yet another embodiment, the additive if present is less than 50% by weight with respect to the total weight of the composition.

According to yet another embodiment, the composition is a bio-accessible composition.

According to another aspect of present invention, there is disclosed a process for preparing a composition of mango extract rich in mangiferin comprising the steps of:
a. dissolving mangiferin in purified water;
b. dissolving separately a hydrophilic carrier and an alkalizer in purified water;
c. mixing solutions of step a. and b. and passing it through a high pressure homogenizer to form uniform dispersion.
d. spray drying the dispersion of step c.

According to an embodiment of present invention, passing said mixed solution through homogenizer in step c is performed preferably for about 15-30 minutes.

According to another embodiment of present invention, passing said mixed solution through homogenizer in step c is performed more preferably for about 15-25 minutes.

According to another embodiment of present invention, passing said mixed solution through homogenizer in step c is performed most preferably for about 15-20 minutes.

According to another embodiment of present invention, said spray drying in step d. is performed at an inlet temperature from about 100° C. to about 180° C. and an outlet temperature from about 30° C. to about 90° C., preferably inlet temperatures from about 110 to about 150° C. and outlet temperatures from about 30 to about 60° C.

According to an embodiment of present invention, said spray drying in step d. is performed preferably at an inlet temperature from about 110° C. to about 150° C. and an outlet temperature from about 30° C. to about 60° C.

According to yet another embodiment of present invention, said hydrophilic carrier is a natural hydrophilic plant-based carrier.

According to further embodiment of present invention, said hydrophilic carrier is selected from gum arabic powder, xanthan gum, modified starch powder, and combinations thereof.

In an embodiment, said hydrophilic carrier is gum Arabic powder.

In another embodiment, said alkalizer is selected from calcium oxide, calcium chloride, sodium carbonate, lithium carbonate, potassium carbonate, sodium hydrogen carbonate, lithium hydrogen carbonate, potassium hydrogen carbonate, disodium hydrogenphosphate, sodium citrate, dipotassium hydrogenphosphate, sodium acetate, sodium lauryl sulphate, polacriline potassium, calcium hydroxide, meglumine, magnesium aluminometasilicate and magnesium oxide or combination thereof.

In a preferred embodiment, the alkalizer is magnesium oxide with at least one other alkalizer as exemplified above.

In yet another embodiment, the mangiferin in step a. is dissolved in 80% of purified water.

In a further embodiment, the carrier in step b. is dissolved in 20% of purified water followed by dissolving in the said solution an optional further carrier, the alkaliser and the emulsifier.

In another embodiment, the amount of mangiferin per dosage form is about 300 mg.

In an embodiment, wherein the composition is in oral dosage form.

In further embodiment, the oral dosage form is selected from a buccal strip, bar, gummies, a chewing gum, a tablet, a capsule, an emulsion, a suspension, an oral spray, effervescent, dissolvable granules or powder, a sachet, and a clear beverage.

In another embodiment, the buccal strip is an orally dissolvable or dispersible buccal strip. In another embodiment, the oral dosage form is a tablet.

In yet another embodiment, the oral dosage form is a capsule.

In yet another embodiment, the mangiferin acts as a catechol-o-methyltransferase (COMT) inhibitor.

According to another aspect of present invention, there is provided a therapeutic method for improving working memory, selective attention and executive function and enhancement of cognitive abilities comprising the step of administering the composition as discussed above.

According to an embodiment of present invention, the dosage of the mangiferin administered is about 300 mg per day.

According to yet another embodiment, the dosage of the mangiferin administered is for about 7 days.

The present inventors have developed a proprietary encapsulation technology where in the composition will maintain in-situ alkaline environment and minimizes the precipitation issues of mangiferin in the acidic environment. Mangiferin is emulsified within the hydrophilic carrier for example, gum Arabic solution to form a carrier matrix in which mangiferin and the alkalizer are embedded.

A natural hydrophilic plant-based carrier along with a selected alkalizer is being used in the encapsulation matrix which subsequently acts as a platform to ensure the alkaline environment with better solubility of bioactive across the physiological pH range.

### EXAMPLES

In light of the foregoing description, the specific non-limiting examples presented below are for illustrative purposes and not intended to limit the scope of the invention in any way.

According to a representative example, the composition of present invention may comprise the following constituents in the following amounts:

| Sr. No | Ingredient | % W/W |
|---|---|---|
| 1 | Mango Extract (Std. to Mangeferin) | 50-70 |
| 2 | Carrier 1 | 20-30 |
| 3 | Carrier 2 | 5-15 |
| 4 | Alkalizer | 1-5% |
| 5 | Emulsifier | 1-3 |

In a preferred example, the composition of present invention may comprise the following constituents in following amounts:

### Example 1

| Sr. No | Ingredient | | | % W/W |
|---|---|---|---|---|
| 1 | Mango Extract | (Std. | to | 60.00 |
| | Mangiferin) | | | |
| 2 | Gum Arabic Powder | | | 20 |
| 3 | Lysine | | | 15 |
| 4 | Magnesium Oxide | | | 4.5 |
| 5 | Tween-80 | | | 0.5 |

### Process Description:

The Mangiferin is completely dissolved into 80% Purified water in Step 1. Gum Arabic Powder was separately dissolved into 20% Purified water followed by addition of Lysine, Magnesium Oxide and Tween 80 in Step 2. Then, the solution of Step 1& step 2 were mixed together and passed through high pressure homogenizer at specific time for 18 min to form uniform dispersion. Finally, the dispersion of step No-3 was spray dryed using specified parameters Inlet temperature—110° C.-150° C. outlet temp. 30° C.-60° C.

### Assessment of Efficacy and Safety of Present Invention for Cognitive Function:

A randomized, double-blind, placebo-controlled study was designed to evaluate the efficacy and safety of Present invention for enhancement of cognitive abilities in healthy adults. The study was initiated after approval from institutional ethics committee, informed consent from participants and after getting it registered in the Clinical Trials Registry—India (CTRI/2023/02/050063 dated 13 Mar. 2023).

This study was conducted at a single study centre in compliance with the approved protocol, requirements of the ICMR ethical guidelines, the International Council for Harmonization of Technical Requirements for Pharmaceuticals for Human Use (ICH-GCP) (Step 5) ‘Guidance on Good Clinical Practice’ (E6 R2), NDCT Rule (The New Drugs and Clinical Trials, 2019) and Declaration of Helsinki.

A total of 60 healthy adults were recruited using opportunity sampling based on specified inclusion and exclusion criteria and were randomized into two groups in the ratio of 1:1 with equal number of subjects in Present invention and placebo groups. The double-blind study design ensured that both participants and investigators were unaware of the intervention assignments throughout the study duration. The blinding process in this study was further fortified by the incorporation of unique subject IDs and randomization codes. Each participant was assigned a distinct identification number, ensuring confidentiality and privacy throughout the study. The randomization codes, generated by an independent biostatistician, were then linked to these IDs. Present invention and placebo group subjects received their respective capsule products to be taken at a dose of 300 mg per day for 7 days. Cognitive assessments were carried out on Day 1 as well as on Day 7 after the last dose.

Methods: A randomized, double-blind, placebo-controlled clinical study was designed to study the efficacy and safety of present invention. Healthy subjects who regularly played virtual/mobile/computer/laptop game online or offline were asked to consume a capsule containing 300 mg of present invention per day for 7 days. Cognitive ability tests, that were a part of the NIMHANS Neuropsychological Battery and Auditory verbal learning tests were used to assess the cognitive health effects. Psychological stress response, anxiety, mood, subjective working memory, and cortisol levels were also assessed. All assessments were carried out at the baseline and at the end of the study.

Results: Present invention was found to significantly improve the mental speed, attention, working memory, response inhibition, and verbal learning and memory as evident from the results of the multiple cognitive ability tests and from the effect size seen in these tests.

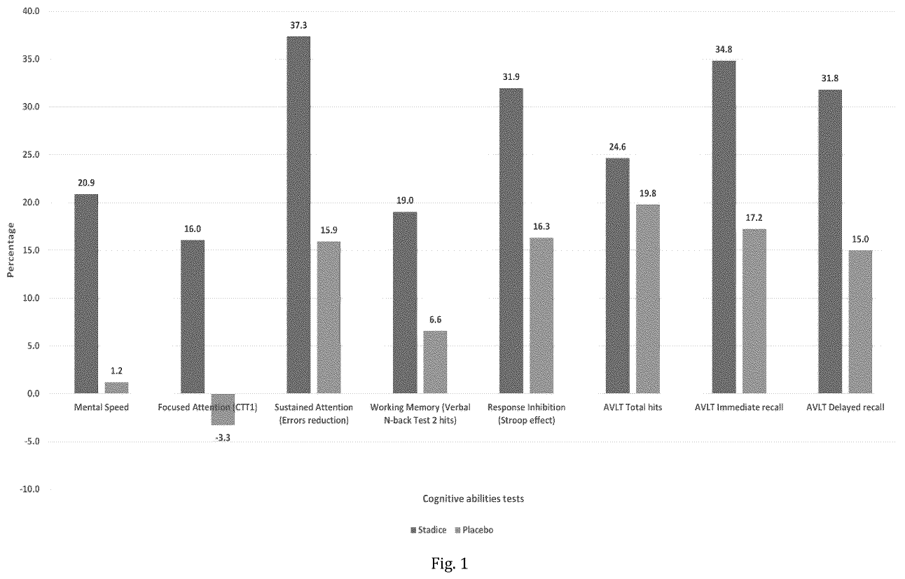

The results of cognitive ability tests in terms of percentage improvement are graphically presented in FIG. 1. Present invention (Stadice®) group showed statistically significant improvement compared to placebo in mental speed (20.9% vs 1.2%, p<0.05) as assessed by DSST, focused attention (16.0% vs −3.3%, p<0.05) as assessed by colour trails test 1, sustained attention (37.3% vs 15.9%, p<0.05) as assessed by errors reduction in Digit vigilance test, response inhibition (31.9% vs 16.3%, p<0.05) as assessed by Stroop Test, AVLT immediate recall (34.8% vs 17.2%, p<0.05) and AVLT delayed recall (31.8% vs 15.0%, p<0.05) as assessed using AVLT. Present invention (Stadice®) group exhibited statistically significant (p<0.05) improvement (19.0%) from the baseline, in case of working memory as assessed using Verbal N-back test 2-hits compared to the placebo group, which showed a minimal change of 6.6% improvement. In the AVLT, significant improvement from the baseline was observed in AVLT trial 1 (47.3%, p<0.001), trial 2 (35.4%, p<0.005), trial 3 (17.9, p<0.05), trial 4 (18.5% p<0.05) and total hits (24.6% p<0.01) in the test group.

Additionally, present invention showed beneficial responses in managing psychological stress in terms of handling nervousness, irritability, or mood swings. No safety concerns found in the laboratory safety test parameters as they were within the normal physiological range and no adverse events were reported in this study.

Advantages embodiment of the present invention is as follows:
1. A holistic natural ingredient formula exploring high therapeutic benefits of plant extracts.
2. A clean label solvent free manufacturing process.
3. Targeted as a natural alternative for regulating cognitive health, alertness and other health segments.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope.

We claim:

1. A composition comprising mango extract rich in mangiferin encapsulated within a hydrophilic carrier matrix comprising an alkalizer embedded therein.

2. The composition as claimed in claim 1, wherein the composition comprising at least 50% mangiferin encapsulated within at least 20% a hydrophilic carrier matrix comprising at least 1% an alkalizer embedded therein.

3. The composition as claimed in claim 1, wherein said hydrophilic carrier is a natural hydrophilic plant-based carrier.

4. The composition as claimed in claim 1, wherein said hydrophilic carrier is selected from gum arabic powder, xanthan gum, modified starch powder, combinations thereof.

5. The composition as claimed in claim 4, wherein said hydrophilic carrier is gum Arabic powder.

6. The composition as claimed in claim 5, wherein said alkalizer is selected from calcium oxide, calcium chloride, sodium carbonate, lithium carbonate, potassium carbonate, sodium hydrogen carbonate, lithium hydrogen carbonate, potassium hydrogen carbonate, disodium hydrogenphosphate, sodium citrate, dipotassium hydrogenphosphate, sodium acetate, sodium lauryl sulphate, polacriline potassium, calcium hydroxide, meglumine, magnesium aluminometasilicate and magnesium oxide or combination thereof.

7. The composition as claimed in claim 1, wherein said composition further comprises an emulsifier.

8. The composition as claimed in claim 7, wherein said composition if present, is present in an amount about 0.5% to about 5% (w/w) of an emulsifier.

9. The composition as claimed in claim 8, wherein the emulsifier is selected from Polysorbate 80, sunflower lecithin and combinations thereof.

10. The composition as claimed in claim 1, wherein the composition further comprises an additive.

11. The composition as claimed in claim 10, wherein the additive, if present is present in the form selected from thickeners, fragrances, pearlescent agents, preservatives, anionic or non-ionic or cationic or amphoteric polymers, proteins, protein hydrolysates, fatty acids, vitamins, panthenol, vegetable, mineral or synthetic oils, gelling agents, antioxidants, solvents, fragrances, fillers, coloring materials and combinations thereof.

12. The composition as claimed in claim 10, wherein the additive if present is less than 50% by weight with respect to the total weight of the composition.

13. The composition as claimed in claim 1, is a bio-accessible composition.

14. A process for preparing a composition of mango extract rich in mangiferin comprising the steps of:
a. dissolving mango extract rich in mangiferin in purified water;
b. dissolving separately a hydrophilic carrier and an alkalizer in purified water;
c. mixing solutions of step a. and b. and passing it through a high pressure homogenizer to form uniform dispersion.
d. spray drying the dispersion of step c.

15. The process as claimed in claim 14, wherein passing said mixed solution through homogenizer in step c is performed preferably for about 15-30 minutes.

16. The process as claimed in claim 14, wherein passing said mixed solution through homogenizer in step c is performed more preferably for about 15-25 minutes.

17. The process as claimed in claim 16, wherein passing said mixed solution through homogenizer in step c is performed most preferably for about 15-20 minutes.

18. The process as claimed in claim 14, wherein said spray drying in step d. is performed at an inlet temperature from about 100° C. to about 180° C. and an outlet temperature from about 30° C. to about 90° C., preferably inlet temperatures from about 110 to about 150° C. and outlet temperatures from about 30 to about 60° C.

19. The process as claimed in claim 14, wherein said spray drying in step d. is performed preferably at an inlet temperature from about 110° C. to about 150° C. and an outlet temperature from about 30° C. to about 60° C.

20. The process as claimed in claim 14, wherein said hydrophilic carrier is a natural hydrophilic plant-based carrier.

21. The process as claimed in claim 20, wherein said hydrophilic carrier is selected from gum arabic powder, xanthan gum, modified starch powder, and combinations thereof.

22. The process as claimed in claim 21, wherein said hydrophilic carrier is gum Arabic powder.

23. The process as claimed in claim 14, wherein said alkalizer is selected from calcium oxide, calcium chloride, sodium carbonate, lithium carbonate, potassium carbonate, sodium hydrogen carbonate, lithium hydrogen carbonate, potassium hydrogen carbonate, disodium hydrogenphosphate, sodium citrate, dipotassium hydrogenphosphate, sodium acetate, sodium lauryl sulphate, polacriline potassium, calcium hydroxide, meglumine, magnesium aluminometasilicate and magnesium oxide or combination thereof.

24. The process as claimed in claim 14, wherein the mangiferin in step a. is dissolved in 80% of purified water.

25. The process as claimed in claim 14, wherein the carrier in step b. is dissolved in 20% of purified water followed by dissolving in the said solution an optional further carrier, the alkaliser and the emulsifier.

26. The composition as claimed in claim 1, wherein the amount of mangiferin per dosage form is about 300 mg.

27. The composition as claimed in claim 26, wherein the composition is in oral dosage form.

28. The composition as claimed in claim 27, wherein the oral dosage form is selected from a buccal strip, bar, gummies, a chewing gum, a tablet, a capsule, an emulsion, a suspension, an oral spray, effervescent, dissolvable granules or powder, a sachet, and a clear beverage.

29. The composition as claimed in claim 28, wherein the buccal strip is an orally dissolvable or dispersible buccal strip.

30. The composition as claimed in claim 28, wherein the oral dosage form is a tablet.

31. The composition as claimed in claim 28, wherein the oral dosage form is a capsule.

32. The composition as claimed in claim 1, wherein the mangiferin acts as a catechol-o-methyltransferase (COMT) inhibitor.

33. A therapeutic method for improving working memory, selective attention and executive function and enhancement of cognitive abilities comprising the step of administering the composition as claimed in claim 1.

34. The method as claimed in claim 33, wherein the dosage of the mangiferin administered is at least about 300 mg per day.

35. The method as claimed in claim 34, wherein the dosage of the mangiferin administered is for at least about 7 days.
